(19) United States
(12) Patent Application Publication
(10) Pub. No.: US 2014/0378828 A1
(43) Pub. Date: Dec. 25, 2014

(54) HIP ARTHROPLASTY METHOD AND WORKFLOW

(71) Applicants: Brad L. Penenberg, Los Angeles, CA (US); Wenchao Tao, Los Angeles, CA (US)

(72) Inventors: Brad L. Penenberg, Los Angeles, CA (US); Wenchao Tao, Los Angeles, CA (US)

(21) Appl. No.: 14/481,810

(22) Filed: Sep. 9, 2014

Related U.S. Application Data
(63) Continuation-in-part of application No. 13/633,799, filed on Oct. 2, 2012, now Pat. No. 8,831,324.
(60) Provisional application No. 61/895,360, filed on Oct. 24, 2013.

(51) Int. Cl. A61F 2/46; A61B 19/00; A61B 6/00; A61B 6/12; A61F 2/34

(52) U.S. Cl. CPC A61F 2/4657; A61B 6/12; A61F 2/34; A61F 2/4609; A61B 6/4441; A61B 6/5258; A61B 6/5241; A61B 19/5244; A61B 6/468; A61F 2002/4668; A61F 2002/4659; A61B 2019/5265; A61B 2019/5238
USPC 600/424

(57) ABSTRACT

A surgical method and workflow to improve the efficiency of a surgical procedure by intraoperatively acquiring a digital radiographic image, processing the digital radiographic image, and using information based on the radiographic image to make adjustments during the surgical procedure. A checklist of parameters may be displayed so that the surgeon can confirm all considerations have been made for the surgical procedure.

### CROSS-REFERENCES TO RELATED APPLICATIONS

This application is a continuation-in-part to U.S. patent application Ser. No. 13/633,799, filed Oct. 2, 2012. This application also claims priority to U.S. Provisional Patent Application Ser. No. 61/895,360, filed Oct. 24, 2013. These references are incorporated herein by this reference thereto.

### TECHNICAL FIELD

This invention relates to a surgical method and a workflow or protocol to make a surgical procedure more efficient using digital radiographic imaging.

### BACKGROUND

In conducting certain surgical procedures, such as total hip arthroplasty (“THA”), the surgeon relies on certain instrumentation to help guide the proper placement of the prosthesis. Unfortunately, even with current technology, any surgeon is likely to admit that reproducibility of implant positioning is not adequate. Trying to get a quality X-ray of a patient using various imaging techniques can be time consuming and potentially exposes the patient to excessive radiation. The surgeon has to wait as the film is taken to another location for processing and brought back for review. If there are any problems with the film, the X-ray must be taken again. As such, there is no convenient intraoperative technique available for achieving perfect prosthesis placement every time.

For the foregoing reasons there is a need for a system that allows the surgeon to acquire a high-quality radiographic image that can be processed in the operating room so as to minimize the overall surgical procedure time and potential excess radiation exposure.

### SUMMARY

The recent introduction of digital imaging technology presents an opportunity to incorporate a new method of acquiring a working, appropriately oriented X-ray and the creation of software to improve the efficiency, precision, and effectiveness of a surgical procedure, such as total hip arthroplasty. The method comprises acquiring a digital radiographic image of a patient, analyzing the radiographic image through a checklist, and making necessary adjustments to the patient or prosthesis based on processed information with minimal interruption in the surgical procedure.

The surgeon can walk through a workflow of choices to guide him through each step of the surgical procedure selected. The workflow instructions guide the surgeon through estimated component placement and a checklist of items to consider during the surgery. For example, in estimating component placement using various bone preparation tools, the invention facilitates the creation of annotations to determine if the broach size and orientation are appropriate. The surgeon can determine acetabular component orientation and the critical parameter of apposition, which is measurable via the software code. Similarly, software enabled assessment of screw position, limb length, and offset can be made.

A trial range of motion is then carried out and, if the hip is stable, the surgeon can now obtain anteroposterior, posteroanterior, and cross pelvis lateral radiographic images. In working through the checklist, the surgeon can reconcile or instruct the software as to right/left side, adjust for optimal contrast, or reconcile patient position parameters based on the preoperative image. He can adjust for pelvic rotation using trans-ischial and mid-sacral lines, mid-symphysis line, or obturator profilometry, reconcile tilt, using a newly described reference factor described below, as compared to this reference preoperative image (X-ray, CT, MRI, ultrasound, or the like), read all parameters, make adjustments as indicated, and repeat films as necessary.

### BRIEF DESCRIPTION OF DRAWINGS

FIG. 1 shows an embodiment of the computer architecture in keeping with the present invention.

FIG. 2 shows an embodiment of an image frame.

FIG. 3 shows an embodiment of a patient information window.

FIG. 4 shows an embodiment of a scanning window.

FIG. 5 shows an embodiment of a digital radiographic image being acquired.

FIG. 6 shows an embodiment of an image quality check window.

FIGS. 7A to 7F and 7H show images in keeping with the present invention in use for total hip arthroplasty.

FIG. 7G shows an example of an ellipse representing the position of an acetabular cup forming a basis for an algorithm in keeping with the present invention.

FIG. 8 shows another embodiment of an image frame of a series of a study.

FIG. 9 shows an embodiment of a computer architecture in keeping with the present invention.

### DETAILED DESCRIPTION OF THE INVENTION

The detailed description set forth below in connection with the appended drawings is intended as a description of presently-preferred embodiments of the invention and is not intended to represent the only forms in which the present invention may be constructed or utilized. The description sets forth the functions and the sequence of steps for constructing and operating the invention in connection with the illustrated embodiments. It is to be understood, however, that the same or equivalent functions and sequences may be accomplished by different embodiments that are also intended to be encompassed within the spirit and scope of the invention.

The present invention is a system and method for a surgical workflow. It allows radiographic images, such as X-rays or CT scans, to be acquired and displayed in digital form on a host computer for immediate review during a surgical procedure. Quality intraoperative (“intra-op”) X-ray images can now be acquired instantly with new digital technology without having to pause an operation while the surgeon waits for hardcopy X-rays to be developed in the traditional way. With the present system, high-quality digital radiographic images can be acquired instantly, soon after the image is taken, rather than waiting for the film development process with traditional X-rays. The digital images can also be archived or forwarded to other medical personnel for further evaluation as required. With immediate acquisition of a high quality radiographic image, the surgeon is able to make the necessary adjustment on the patient or a prosthesis to add precision and efficiency to the surgical procedure.

The surgeon is able to acquire or access radiographic or X-ray images before beginning the surgery (“pre-op”) to conduct a preoperative assessment. By conducting a preoperative assessment, the surgeon is able to better position his patient for a more accurate surgical procedure resulting in a better outcome. Radiographic images can also be stored anywhere and retrieved by the surgeon through a network connection.

Once the proper positioning of the patient has been established, the surgeon can then perform the surgical procedure. For example, to assure a continued understanding of how the pelvis is oriented in space, a goniometric or accelerometric device can be secured to the patient with adhesive on the skin or pinned into the bone. Additionally, during the surgery, an intraoperative radiographic image (“intra-op radiographic images”) may be acquired to assure proper positioning of all components used in the surgery. The readout from the accelerometric device in the known position is confirmed by the X-ray and recorded. The operation proceeds in standard fashion. At the point of obtaining the trial radiograph and applying the software annotations, the patient is positioned such that the electronic readout on the goniometric or accelerometric device is as close as feasible under the circumstances to the readout when the earlier “positioning X-ray” was taken.

Before the surgery is complete, additional intraoperative radiographic images may be acquired to assure proper positioning of all components used in the surgery. The need for these additional intraoperative radiographs can be reduced, however, by applying a correlation between the angular position of the pelvis (around the major axis of the pelvic inlet) observed during surgery with an angular adjustment or offset that the surgeon will need to make to the abduction angle, as discussed in greater detail below.

This surgical workflow may be embodied in a computer software configured to facilitate making measurements on a radiographic image so as to determine and/or calculate the proper positioning of the patient before (“pre-op”), during (“intra-op”), and after (“post-op”) surgery. In order to utilize the workflow, a radiographic image of the patient must first be acquired.

### Image Acquisition

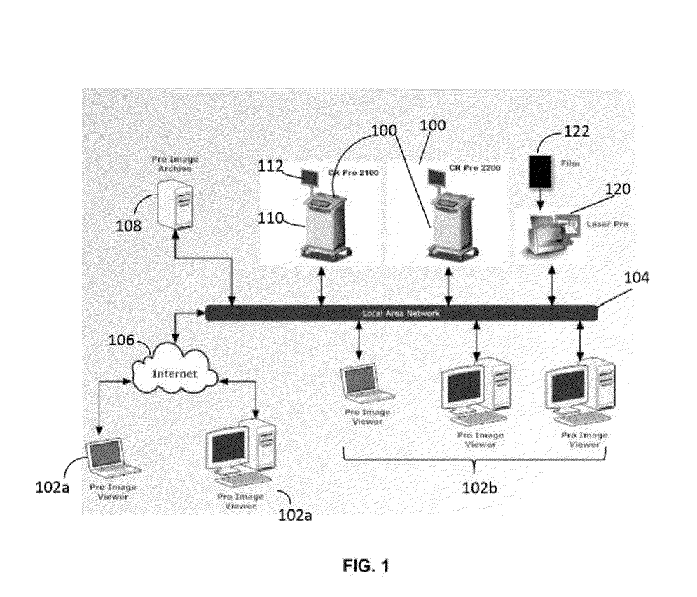

Digital radiographic images can be acquired via digital radiography or computed radiography. For example, as shown in FIG. 1, in computed radiography, a radiological device 100 scans erasable phosphor plates exposed to X-rays and transmits the images to an imaging onboard computer 110 (either through a wired or wireless connection) for review, archiving, or forwarding to other facilities 102a, 102b through a local area network 104 and/or through the Internet 106 for further evaluation and/or archiving 108. In some embodiments, a scanner 120 may scan existing X-ray files 122 and convert them to digital radiographic images. Acquired images can be saved in a variety of formats, such as tiff, jpeg, png, bmp, dicom, and raw formatted images for both reading and writing.

Digital radiography offers numerous advantages over traditional X-ray methods. For example, digital radiography generates low radiation levels, specifically, below levels required by traditional X-ray machines to capture a detailed image on radiographic film 122. In addition, a radiographic image 500 can be viewed much quicker than with traditional X-ray film due to the reduced image acquisition time. Therefore, overall exposure to radiation is reduced due to the lower levels and shorter exposure times.

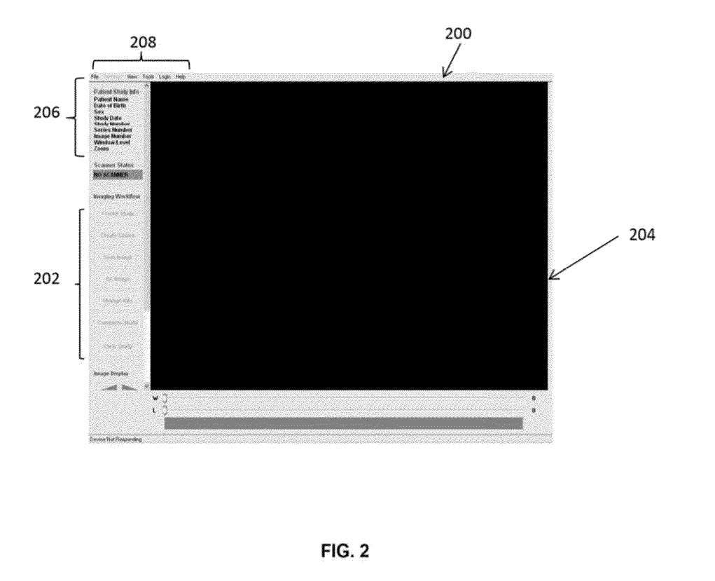

As shown in FIGS. 2-8, once a digital radiographic image 500 has been acquired, the radiographic image 500 can be processed and optimized by a computer 110, or any other computing device, such as a tablet, a smart phone, and the like. The computer 110 will display on a display device 112 (such as a monitor, television, screen, tablet, etc.) a main screen or main window 200 providing workflow action buttons 202, an image frame 204 to display the radiographic image 500, an information frame 206 displaying the information associated with the radiographic image displayed, and typical menu items 208.

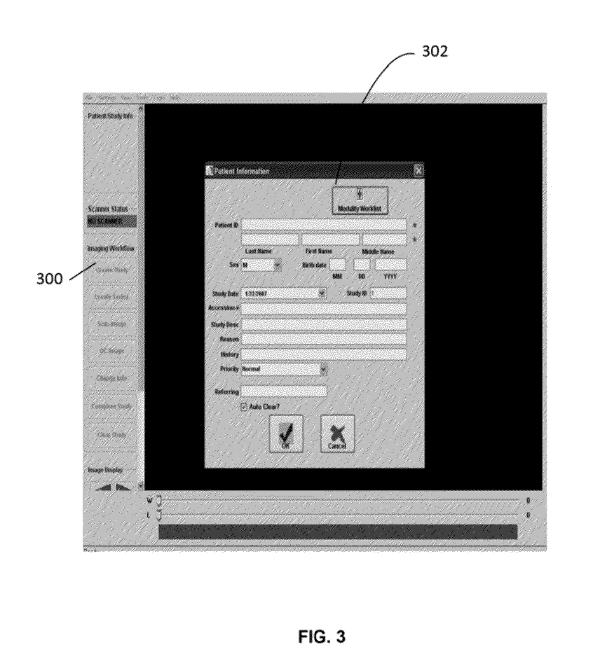

The workflow 202 steps may comprise creating a study, creating a series, scanning an image, performing quality control on the image, changing information, completing the study, and clearing the study. Creating a study begins with entering information related to the patient, the patient's medical condition, a recommended medical procedure, and any other information relevant to the patient and the condition being treated or diagnosed. As shown in FIG. 3, a create study button 300 can be provided to begin this process. A patient information window 302 may be provided with various fillable fields to input the relevant information. Once a study is created, an acquired radiographic image 500 can be uploaded and saved to that study.

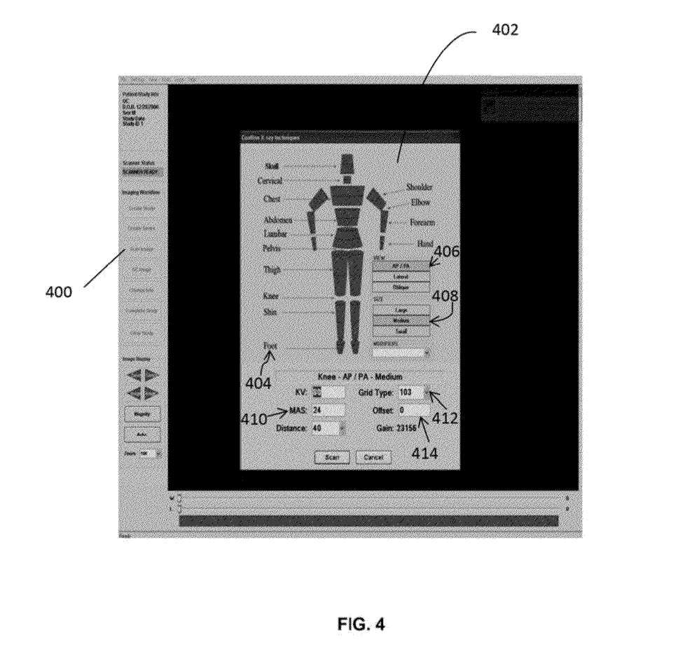

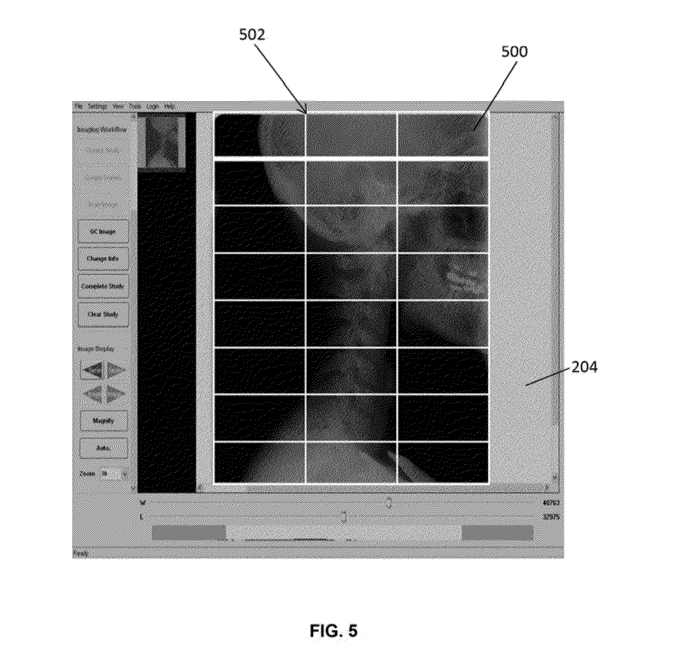

Scanning an image allows the user to acquire a radiographic image of a particular body part (i.e. anatomical structure) for processing. As shown in FIG. 4, a scan image button 400 may be provided, actuation of which opens a scanning window 402 that allows the user to select such configurations as a particular body part or region 404 to be scanned, the view of the scan 406, the patient's size 408, the particular technique used 410, the grid type 412, the offset 414, and the like. Selecting a body part may involve selecting the corresponding body part of an image displayed on the image frame, or selecting the body part from a list of body parts. Once the scan is initiated, the images may be displayed line by line in the image frame 204 as it is being scanned as shown in FIG. 5. In some embodiments, a grid 502 may be provided. Using the grid 502, the image orientation can be verified and adjusted. For example, the image 500 can be rotated by any degree, flipped, inverted, or otherwise adjusted, and processed further as discussed below.

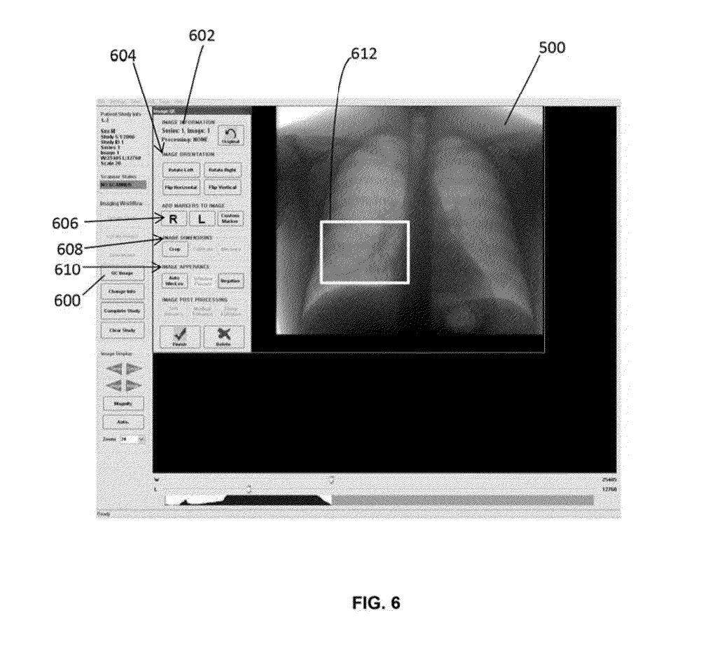

Once the image 500 has been acquired, the user can perform a quality control (QC) check on the image. As shown in FIG. 6, a layout template or a QC image button 600 may be provided, actuation of which opens an image QC window 602 providing a number of features to improve the quality and layout of the radiographic image 500 acquired. For example, the image QC window 602 may provide features to orient the image 604, add markings 606 to the image, annotate the image 607, change the image dimensions and take measurements or sizing on the image 608, change the appearance of the image 610, and the like.

Therefore, using the image QC window 602, the user can process, modify, and interact with the image 500, such as rotating to the left, rotating to the right, flipping horizontally, flipping vertically, cropping, zooming, magnifying, moving or panning the image, changing the window width, inverting the image to a negative image, adjusting the level (brightness and contrast) of the image, adding or creating markings to indicate various information about the image, adding measuring lines to determine lengths, distances, angles, and the like. Additional features can be added, and any feature can be removed and added back again so as to configure the layout template according to the user's needs.

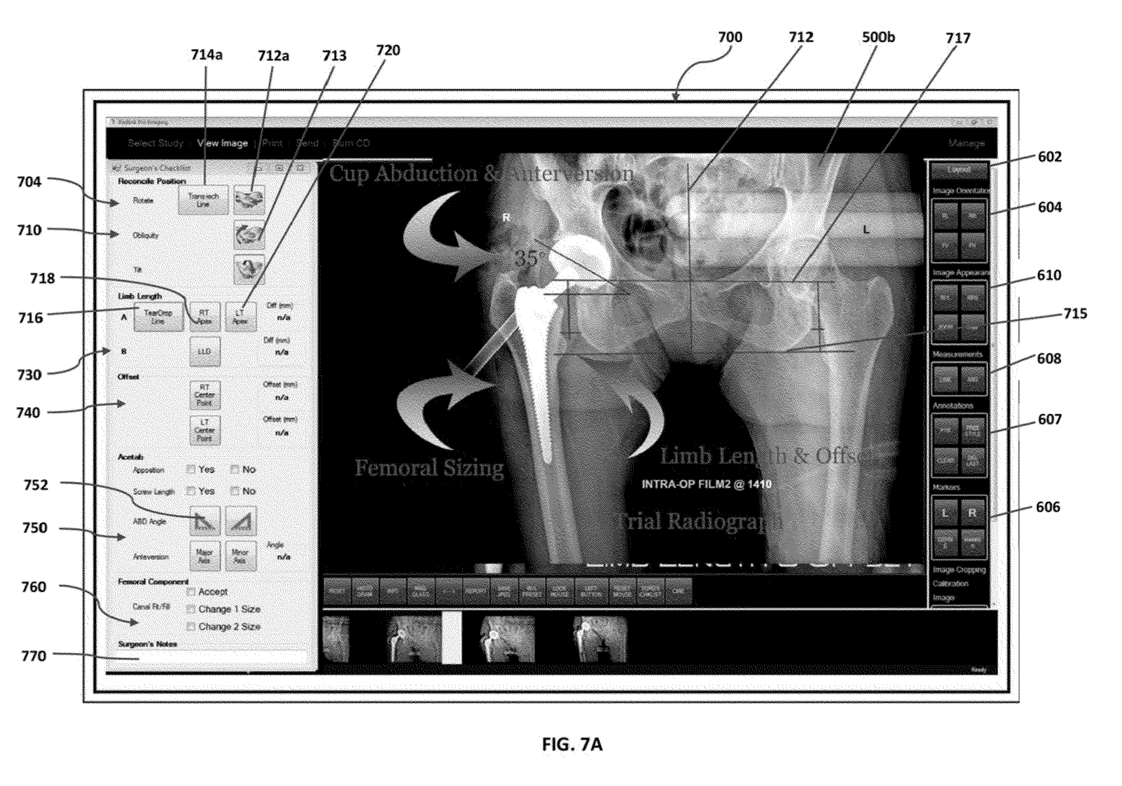

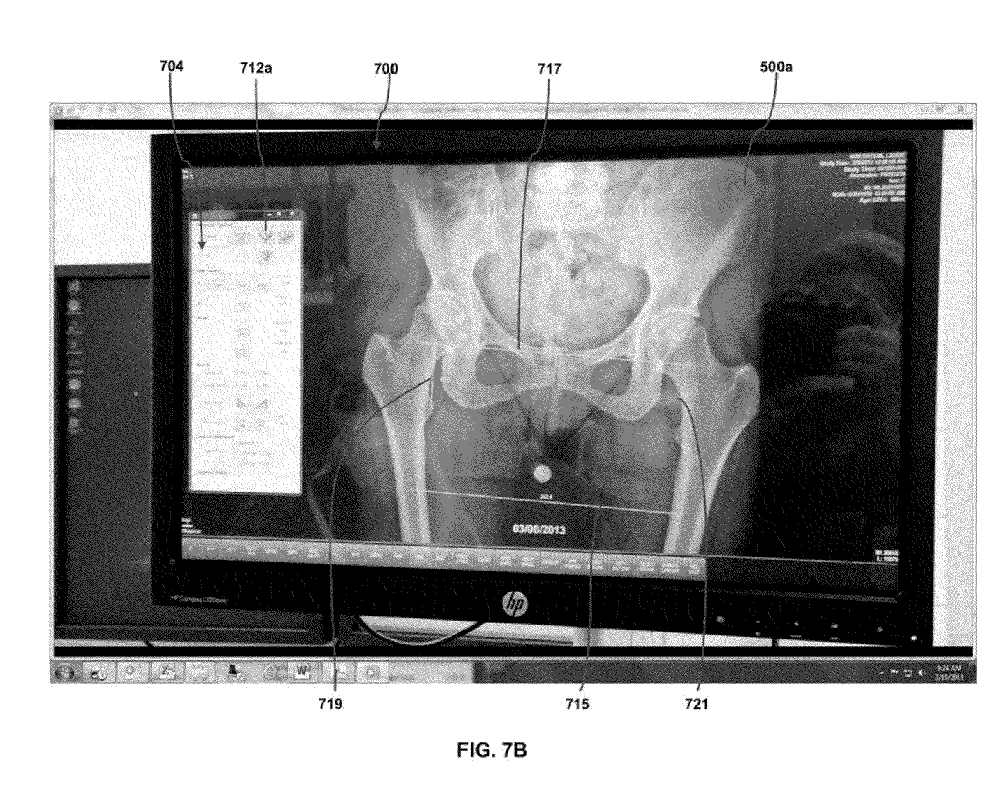

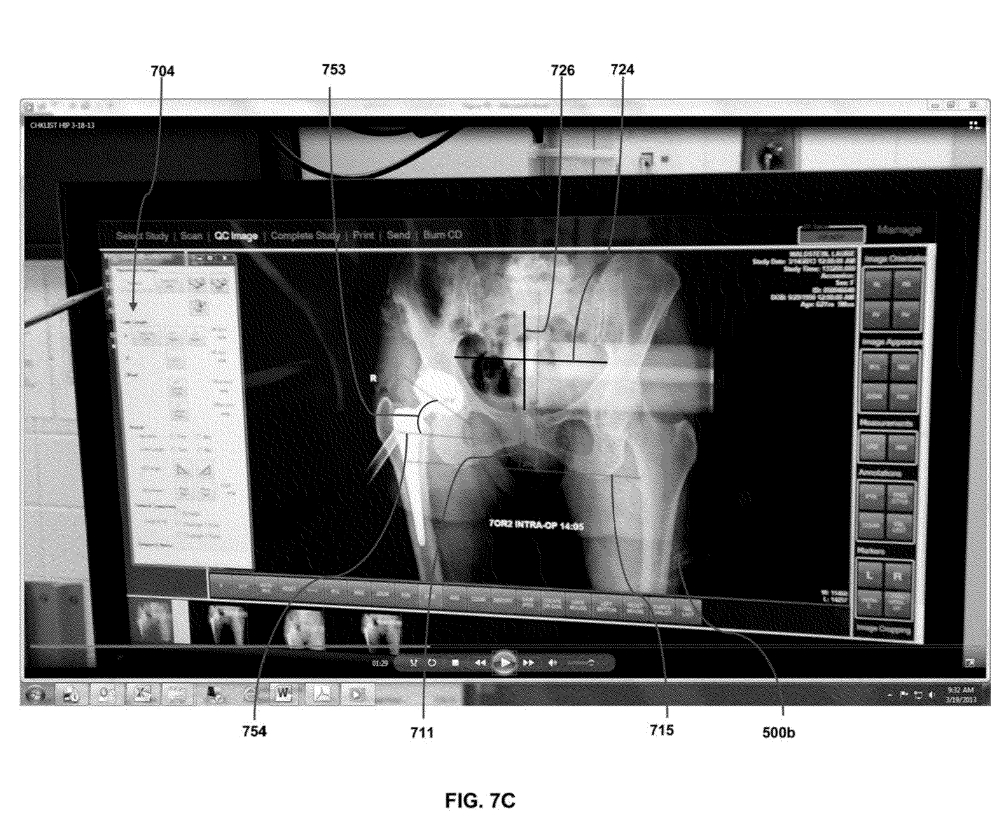

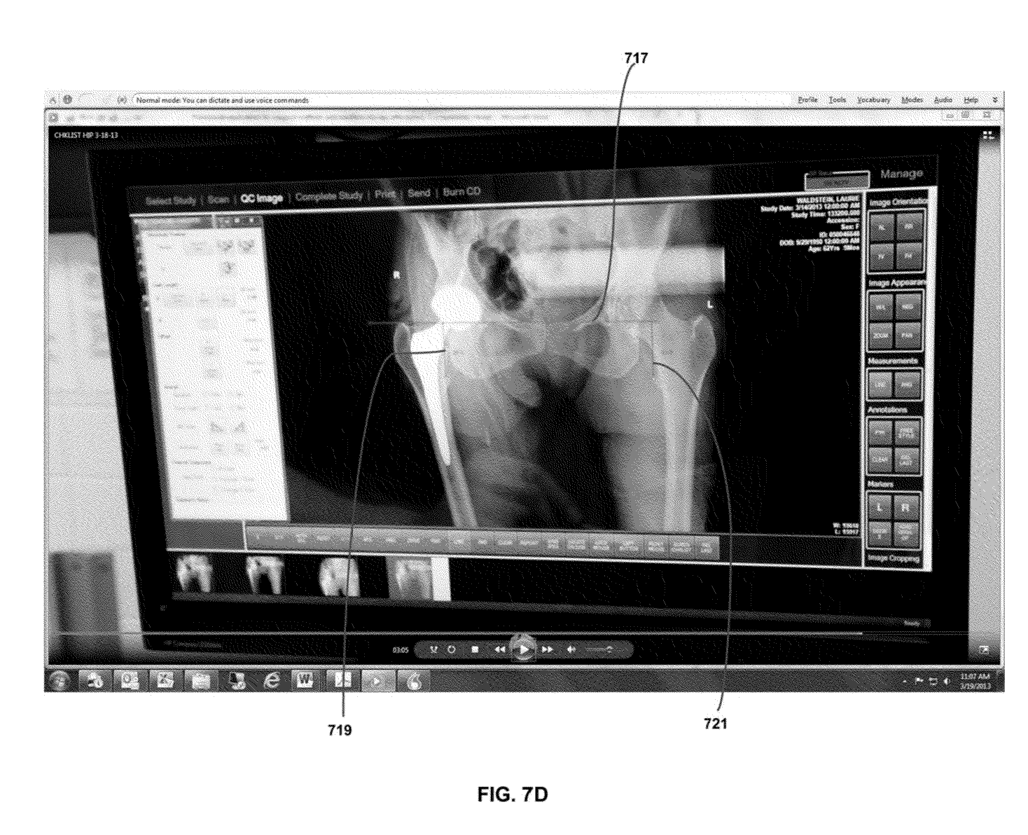

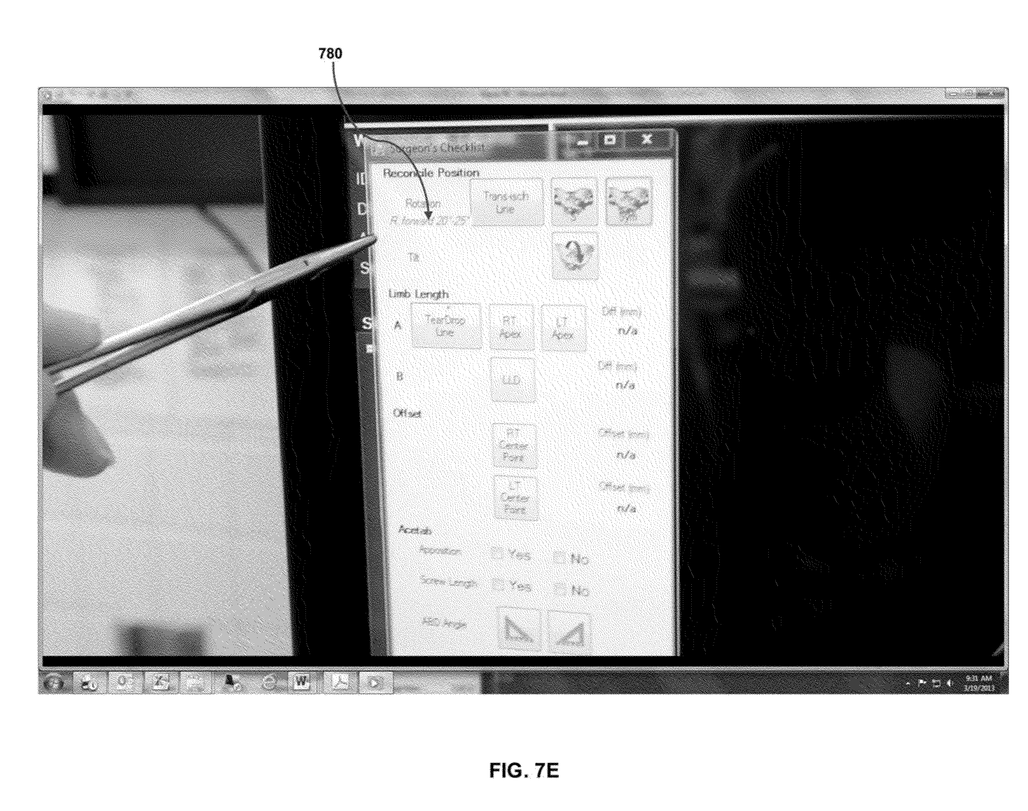

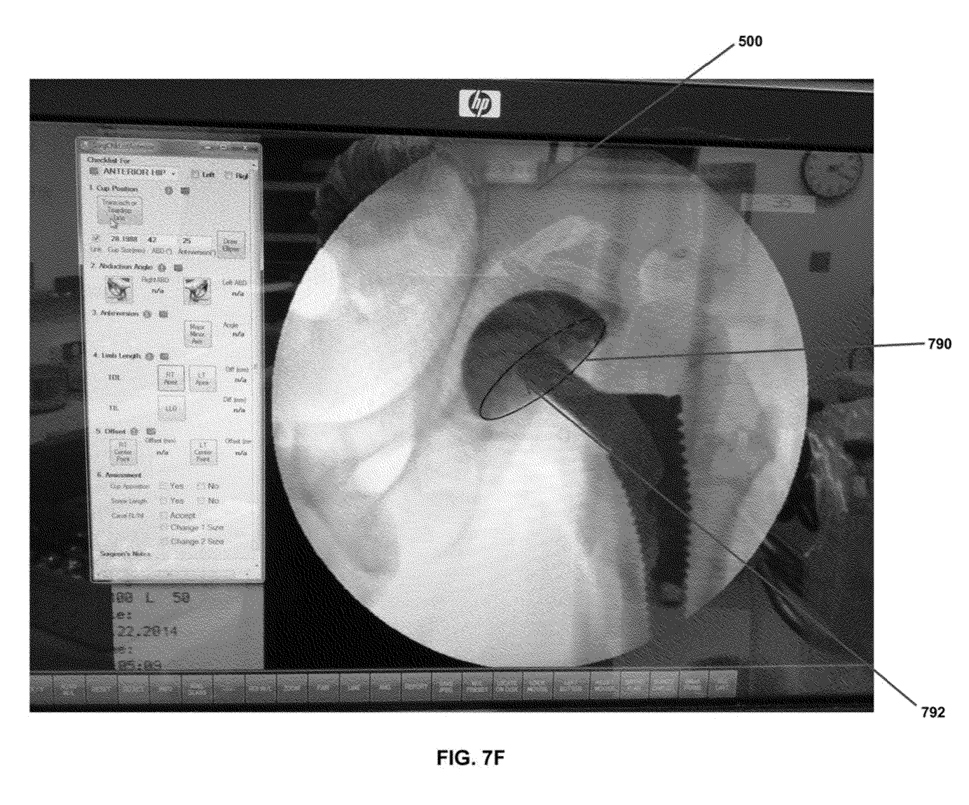

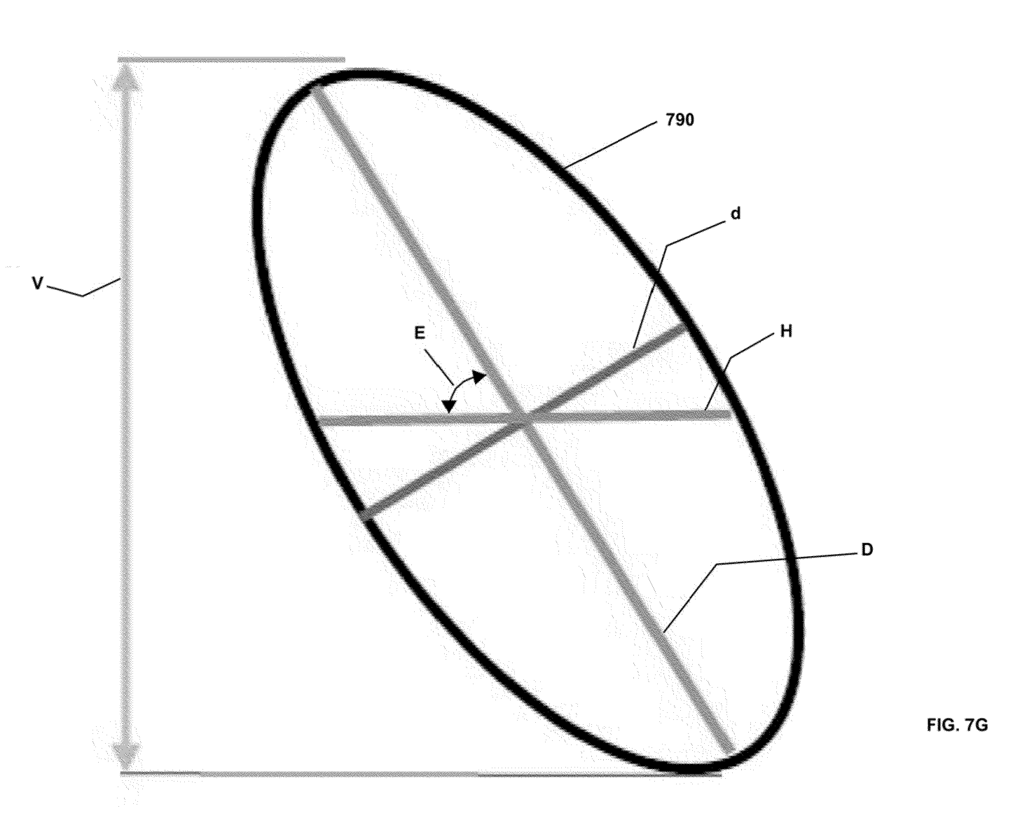

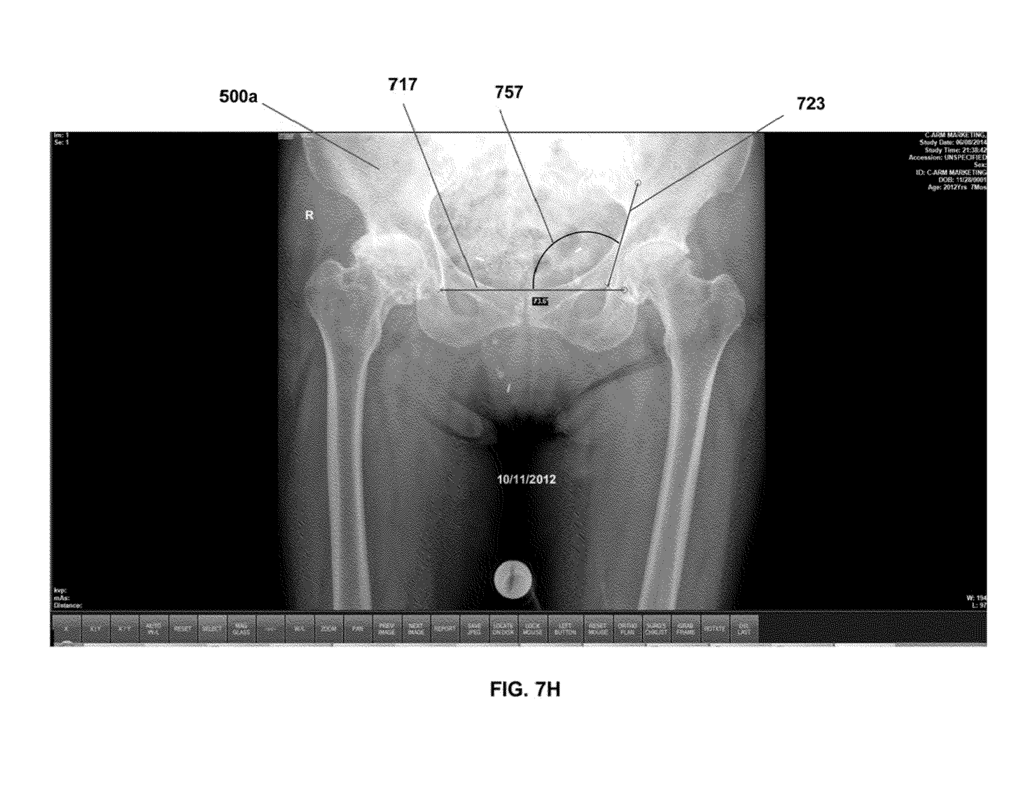

In some embodiments, processing, modifying, and interacting with the image 500 can be done on the image frame 204 instead of with the image QC window 602. For example, the display device 110 showing the radiographic image 500 may be a touchscreen device 700 as shown in FIG. 7A. The user may use his fingers or a pointing device or any tool 702 to make gestures on a touchscreen 700 to effectuate the desired feature and view parameters that would assist the surgeon during the surgical procedure. For example, if a radiographic image 500 is not properly aligned in the image frame 204, the user may tap or double tap the touchscreen 700 on the scanned image 500 and cause it to automatically rotate into the proper orientation. Gridlines 502 may be displayed on the image frame 204 as a guide for proper alignment. Alternatively, the user can place one or more fingers on the image frame 204 and make a rotating gesture to cause image 500 to rotate in the direction of the gesture. In some embodiments, the user may input specific values as the precise measurement for modification. For example, the user may indicate that the radiographic image 500 is to be rotated by a specified angle of rotation.

Once the radiographic image 500 is properly oriented, the anatomical structures shown in the image 500 can be marked with markings, such as symbols, annotations, measurements, and the like, and any combination thereof. Taking measurements can be done in a similar fashion. As shown in FIG. 7A, actuation of any of the features in the image QC window 602 may open a tools window 704 containing a checklist (surgeon's checklist) with electronic tools that are specific for the feature actuated. For example, there may be a pelvic positioning section 710 comprising tools to allow the surgeon to correctly determine the pelvic position. There may be a section for limb length measurements 730 to determine whether the limb length of the leg operated on is within an acceptable range. There may be an offset section 740 to determine whether the offset of the prosthetic is within an acceptable range. There may be an acetabulum section 750 to determine whether the acetabular abduction angle and anteversion are within acceptable ranges. There may be a femoral component section 760 determine whether the febrile component is within an acceptable range. Finally, there may be a notes section 770 for the surgeon to input any notes and comments. Actuating any of the tools in the tools window 704 will allow the surgeon to create appropriate lines and/or angles on the radiographic image to take the proper measurements. In some embodiments, actuating any of the tools in the tools window 704 automatically creates the proper line or angle and the surgeon can move the line or angle to its proper position as well as change the length or angle. In some embodiments, actuation of any of the tools in the tools window 704 primes the screen so that when the surgeon touches the screen, the surgeon can draw the line angle any starting point on-screen. In the preferred embodiment, once the surgeon generates the first line, all subsequent lines and angles are keyed off of or in relation to the first or any previously drawn lines or angles as described below. Any line or angle already drawn can be modified to change the length and/or angle.

Based on the markings on the radiographic image 500, the surgeon or other medical personnel can make adjustments before and during the surgical procedure, such as adjusting the position of the patient, the patient's body part, a prosthetic, a surgical tool, and the like to achieve a desired position, thereby eliminating any delay and interruption during the surgical procedure. The adjustments to the patient may be based on any differences between an intra-op image 500b and a predetermined or expected standard. For example, the standard may be based on a pre-op image 500a, an image of the non-operative side, a prior image, a known value, a calculable value, a constant, and the like. So, in one example, adjustments to the patient, prosthetic, and/or medical device may be based on any differences between a pre-op image 500a and an intra-op image 500b. Adjustments may be made so that the intra-op image 500b corresponds as closely as possible to the pre-op image 500a. In another example, if the anatomical structure at issue has a bilateral counterpart, then the intra-op image 500b can be compared to the counterpart anatomical structure that is not being operated on (the non-operative side).

In another example, measurements of a particular anatomical structure depicted in the images 500b may be expected to have a specific value. If the intra-op image 500b shows that the anatomical structure has measurements different from the standard, then the patient may be adjusted until the intra-op image 500b results in the standard value.

Derivatives may be used to calculate accurate values, such as reference lines for pelvic orientation with correction prompts; diametric ratio to correct for abduction angle; lesser trochanteric profilometry to correct for the usual errors in limb length and offset measurements by adjusting femoral rotation.

By way of example only, as shown in FIGS. 7A-7H, the capabilities of the invention is demonstrated using total hip arthroplasty as the surgical procedure. Based on the concepts explained in this example with total hip arthroplasty, the invention can be applied to any surgical procedure. FIG. 7A shows an embodiment of the invention with an intraoperative radiographic image 500b, but the inventive concept applies equally regardless of whether the radiographic image 500 is a preoperative image 500a, intraoperative image 500b, or postoperative image.

As shown in FIG. 7B, a preoperative radiographic image 500a of the hip area is acquired and displayed on a touchscreen 700 along with the tools window 704 containing the surgeon's checklist to allow the surgeon to check desired parameters to assure the surgical procedure will be performed efficiently and accurately. Based on the acquired pre-op image 500a, the surgeon must identify the orientation of the pre-op image 500a to make sure the left L and right R sides of the image are properly displayed and the contrast adjusted.

The surgeon may begin by making sure that the pelvis is properly oriented. The surgeon may select a pelvis reconciliation icon so as to ensure that the pelvis is aligned properly. The surgeon may then create a pelvic axis reference line 712.

In many embodiments of the present invention, the surgeon can draw a series of symbols, such as lines and angles, to assure the patient is in the proper orientation and the prosthesis is in the proper position. It is contemplated that this may be done using a touchscreen interface by using a mouse, joystick, touchpad, or the like, to place such points of reference, lines, orientation angles, or other notations on the screen, and thereby into a database for future reference.

Where a touchscreen is used, to facilitate the accuracy of drawing a point or line, the program may allow for a cursor offset. That is, when the surgeon touches the touchscreen, a pointer or cursor will appear and be positioned a predetermined short distance offset from where the surgeon is touching. This is advantageous in the operating room because the surgeon's finger, including his surgical glove, can otherwise get in the way of where the surgeon intends to place the pointer, especially in light of the highly accurate pointer placement that will be desired. Without this offset, the surgeon's finger or other pointing instrument could obstruct his or her view. By having a cursor offset, the surgeon can touch next to where he wants the lines drawn and then drag the cursor to the precise location while never obstructing his or her view of the pointer or cursor or the underlying landmarks on the screen.

Alternatively, the computer system may be equipped with a highly sensitive screen or other two- or three-dimensional detectors (such as using theremins or other virtual movement detectors) so that the surgeon can move the cursor on the screen without touching the screen. As a result, the surgeon may move his finger close to the screen or within the detection field of the two- or three-dimensional detectors. When the system detects the surgeon's finger, it places a cursor or pointer at the corresponding location on the screen. The surgeon may then move the pointer into place by moving his finger or performing other gestures like sweeping or scrolling motions. In doing so, the pointer can be positioned with a high degree of accuracy without the surgeon's finger obstructing the view of the relevant portion of the screen, in fact without touching the screen at all.

Selecting the pelvis reconciliation icon 712a, then touching the touchscreen (or using a mouse, touchpad, etc.), creates a vertical pelvic axis reference line 712. Any line generated on the touchscreen can be moved, lengthened, and shortened. The pelvic axis reference line 712 may be moved so as to align with the mid-sacrum to determine the presence of a pelvic rotation. Significant rotation of the pelvis would render subsequent measurements inaccurate. If the pelvic axis reference line 712, starting at mid-sacrum, passes to one side of the symphysis pubis or pubic symphysis by more than, for example, a centimeter, then the patient's position should be adjusted so that the pelvic line 712 is as close to middle of the pubic symphysis as possible to reconcile pelvic rotation. In some embodiments, the surgeon may draw a pelvic rotation reference line 711. The pelvic rotation reference line 711 is drawn parallel to the pelvic axis reference line 712. If the two lines 711, 712 overlay on top of each other, then the rotational orientation of the hip is correct. If, however, there is later offset between the two lines, as shown in FIG. 7C, then the hip rotation needs to be corrected. The extent of the offset and the amount and direction of rotation may be displayed to inform the surgeon as to the proper corrective measure.

Once it has been determined that the patient is properly aligned on the surgical table, the surgeon may select the trans-ischial line icon 714a, then touch the touchscreen 700 to display a horizontal line, referred to as the trans-ischial line 715. In some embodiments, the surgeon may actually draw the trans-ischial line 715 from any starting point the surgeon wants, such as near the lesser trochanter, and terminate the line anywhere he wishes, such as the opposite lesser trochanter. The trans-ischial line 715, like any other line generated on the radiographic image, can be lengthened or shortened and positioned anywhere. The trans-ischial or trans-ischial tuberosity line 715 may be used to reconcile pelvic rotation. This identifies a horizontal axis orientation in relation to the pelvis. This line 715 may also create a reference for subsequent angular and linear limb length and offset measurements. For example, the trans-ischial line 715 may be positioned at the bottom of the ischium. The trans-ischial line 715 can be lengthened so as to pass beyond the lesser trochanter. Where the line passes through the lesser trochanter on the left and right femurs can help determine the limb length of each leg.

The surgeon may then select a teardrop line icon 716 and touch the touchscreen to generate another horizontal line, referred to as the teardrop line 717. The teardrop line 717 can be positioned at the teardrop. The teardrop line 717 can also determine limb length by measuring the distance between the teardrop line 717 and the lesser trochanter or some other known anatomical landmark. To determine the right limb length, the surgeon may select the right apex icon 718. Touching the screen then automatically creates a right apex line 719 from the point where the surgeon touches, towards and perpendicular to the teardrop line 717. Similarly, to determine the left limb length, the surgeon may select the left apex icon 720 then touch a point on the screen. A left apex line 721 is automatically drawn from the point where the surgeon touches the screen, towards and perpendicular to the teardrop line 717. The distance in some predetermined units may be displayed adjacent to any of the lines discussed thus far and hereafter. This lets the surgeon know, based on a relative distance measurement from a point on the pelvis to a point on the femur, what the current hip joint determined relative limb lengths are before any cutting has begun. The surgeon can use this information during the operation as discussed below. In the example shown in FIG. 7B, the surgeon touches the right and left lesser trochanters, thus creating the right and left lesser trochanteric apex lines 719, 721 from the right and left lesser trochanters, respectively towards and perpendicular to the teardrop line 717.

In some embodiments, when the surgeon has completed a particular stage or set of measurements, the lines created in the process may be automatically removed from the screen, even if only temporarily, to at least partially clear the screen and make the next set of lines and measurements easier to see. Alternatively, a delete or clear screen button 609 may be provided to remove the lines or clear the screen of all annotations, symbols, etc. previously drawn so the surgeon can start new markings on a clean image. For example, when the surgeon has completed measuring the acetabular abduction angle, the screen may automatically clear or allow for a manual clear screen of all lines and annotations pertaining the measuring of the acetabular abduction angle. The surgeon can then begin annotating the next set of measurements, such as limb length or some other parameter on a clear screen.

In some embodiments, one method for assuring that the measurements in the intraoperative images are accurate is to measure the dimensions of the pelvic inlet defined by the pelvic brim. In general, the pelvic inlet has an oval shape with a major diameter or axis and a minor diameter or axis. From the tools window 704, the surgeon may select an icon for the major diameter of the pelvic inlet. Then, by touching the touchscreen 700, a horizontal line, referred to here as the major diameter line 724, will appear. The surgeon can then select and hold the major diameter line and move it relative to the radiographic image so that it is positioned within the pelvic inlet. By holding either end of the major diameter line, the surgeon can lengthen or shorten the major diameter line so as to measure the exact distance of the major diameter of the pelvic inlet at its widest location. The surgeon can then touch an icon for the minor diameter of the pelvic inlet.

Touching the touchscreen 700 then generates a vertical line, referred to here as the minor diameter line 726. Again, the surgeon can move the minor diameter line 726 relative to the image and adjust the length of the minor diameter line so that the minor diameter line is positioned to measure the distance of the minor diameter of the pelvic inlet. The “point-to-point” feature can also be utilized in an effort to accurately target a precise, small bony landmark. The surgeon can use the length of the minor diameter line 726 or the ratio of the lengths of the major diameter line 724 and the minor diameter line 726 as the control that enables intraoperative correction for any pelvic tilt or pelvic rotation in the intraoperative or postoperative radiographic images relative to the preoperative image. This correction accounts for changes in critical parameters such as acetabular component orientation, which could occur if there is significant discrepancy from the control X-ray, as discussed in greater detail below.

Additionally, in some embodiments of the present invention, templating, whether preoperative or otherwise, can be used to assure that all measurements are accurate. Since several factors can affect the overall dimensions on the X-ray, such as the distance between the X-ray machine and the patient, which may vary from image to image, or other scaling effects inherent in the unit hardware and software, such embodiments of the present invention additionally comprise one or more scaling or templating steps to determine a factor or coefficient to correct for this variance in magnification or scaling artifact. The correcting coefficient can be determined from the ratio of a measurement of a landmark, anatomical structure, or linear distance on the radiographic image, on the one hand, to the measurement of the same landmark, anatomical structure, or linear distance taken directly from the patient, on the other hand. By knowing this ratio, the surgeon is able to correct for the inherent magnification of any structures on the radiographic image, including to calculate the actual size of such structures, the actual size of the tools required for the surgery, and the actual size of the prostheses to be used.

In some embodiments, edge detection technology can be used to facilitate the templating process. Edge detection may be important in the initial assessment and progress of corrective surgery.

In some embodiments, a collective rating system may be employed to alert the surgeon as to the accuracy of the operation. For example, in arthroplasty, acetabular component apposition, abduction angle, anteversion, screw position, femoral component sizing, i.e. fit and fill, limb length, offset, the presence of a fracture, and the like are parameters for which accuracy is desired. A red zone/green zone (or any other color coding) or alphanumeric rating system may be used to calculate the proximity to the target parameters. Therefore, during the operation, the surgeon can receive instant feedback as to whether any adjustments are going in the proper direction.

Once the preoperative assessment is complete, the surgeon can perform the surgery, in this example, the total hip arthroplasty. During the surgery, the surgeon can acquire an intraoperative radiographic image 500b as shown in FIG. 7C. With the intraoperative image 500b, the surgeon can acquire the same data and assess the same parameters as discussed above but with the prosthesis in place.

Using the surgeon's checklist in the tools window 704, the surgeon can create his first line to begin taking measurements for the proper prosthesis placement and joint reconstruction. Subsequent lines created will be keyed off of, or referenced from, one of the previously created lines as a reference. For example, after obtaining the intraoperative image or trial radiograph 500b, the surgeon may start with a trans-ischial line 715 to estimate the pelvic rotation and/or limb length. The surgeon can then select the pelvic rotation icon 712a and the pelvic axis reference line 712 will be drawn automatically perpendicular and parallel to the trans-ischial line 715. For example, after selecting the pelvic rotation icon 715a, the surgeon may touch the mid-sacrum above the trans-ischial line 715. The pelvic axis reference line 712 will be drawn from the mid-sacrum towards and perpendicular to the trans-ischial line 715. The surgeon can then check to see if pelvic axis reference line 712 passes through the pubic symphysis and determine whether any adjustments to the pelvic rotation needs to be made. By selecting the rotation reference icon 713, the surgeon can draw a rotation reference line 711 along the pubic symphysis to determine the lateral offset between the pelvic axis reference line 712 and the rotation reference line 711. The unique software feature will perform a calculation based on the offset, or distance from the intersection of the two lines, representing the front and back of the pelvis. A prompt will then appear indicating the need to rotate the table a certain distance in a certain direction in order to match the preoperative pelvic orientation and make the intraoperative measurements valid and most likely to match the postoperative measurements. Other anatomical structures may also be used as reference points for drawing these lines so long as the structures can be used to determine pelvic rotation.

The surgeon may create a teardrop, or inter-teardrop, line 717 to measure the relative limb length. The teardrop line 717 could be created parallel to the trans-ischial line 715 in embodiments in which it is created before the teardrop line 717 is created or using the point-and-drag or point-to-point options.

As shown in FIG. 7D, with the teardrop line 717 created, the surgeon can select the right apex icon 718, activating the “create right apex line”. By touching the screen, the right apex line 719 is drawn automatically from the point where the surgeon touches the touchscreen perpendicular to and towards the teardrop line 717. This step can be repeated for the left apex line 721. To determine limb length, the surgeon will usually select the lesser trochanters as a starting point for the right and left apex lines 719, 721, respectively. The distance from the lesser trochanter to the teardrop line 717 will automatically be displayed so the surgeon can know the relative limb lengths.

The surgeon may create an angle by selecting an angle icon 752 from the surgeon's checklist in the tools window 704. The angle 753 will refer to the teardrop line 717 or the trans-ischial line 715, which was created before the angle 753, so the base of the angle 754 will be automatically created parallel to the teardrop line 717 and/or the trans-ischial line 715. The angle 753 may be used to measure the acetabular abduction angle. Since the desired acetabular abduction angle is generally known, the angle may start out at 45 degrees, for example. The surgeon can then place the angle 753 adjacent to the acetabular component to measure the acetabular abduction angle.

### Tilt Correction Factor for Acetabular Component Orientation

In an embodiment of the present invention, the system calculates for the surgeon an adjustment, or correction factor, for this acetabular abduction angle 753 based on a correlation to one or more of the foregoing intraoperative measurements. One such adjustment calculation, for example, can be derived from the length of the minor diameter line 726 or axis of the pelvic inlet or the eccentricity (e) of the pelvic inlet, calculated from the ratio between the minor 726 and major axes 724 of the pelvic inlet as shown in FIG. 7C. That is, image orientation may be critical to achieving accurate measurements. The changes in cup inclination may be quantifiable according to the magnitude of pelvic tilt variation between pre-, intra-, and postoperative films. The use of a pelvic tilt correction factor can provide standardization while avoiding the need for repeat films.

This is because it has been determined that the measurement of the intraoperative acetabular abduction orientation using digital radiography (DR) may be used to reliably predict postoperative measurement. Such a tilt correction factor for acetabular component orientation may be directly related to the clinical outcome in THA. Various techniques exist to improve implant positioning during THA, but the success rates, radiation exposure, ease of use, and cost have been substantial barriers to widespread acceptance. Applicant uses DR as an alternative for intraoperative imaging and guidance. Compared to chemical image processing, DR provides rapid (2-4 seconds) image acquisition, high quality, and selectively enhanceable images that expose the patient to less radiation than a traditional pelvic radiograph. The image may be generated without the need to move the cassette and therefore may permit an efficient adjustment method assuring correct image orientation.

In an embodiment of the present invention, the preoperative standard supine office radiograph can be used as a reference radiograph for the desired intraoperative image orientation. This postoperative radiograph would be taken in the same setting and may have the same orientation as the preoperative film, and an abduction angle would be measured using the inter-teardrop line as the transverse reference line. The operator can then repeat the radiograph process until a neutral pelvic rotation is present. The pelvic tilt can be generally controlled using a reference vertical inlet dimension 726 or a ratio of the vertical 726 and horizontal 724 inlet dimensions in order to permit mathematical correction for variations in tilt position. In doing so, the surgeon may avoid repeat radiographs. The chosen abduction angle in any individual case is often based on the Lewinnek recommendations. In any case, a final abduction angle can be determined by clinical—not radiographic—parameters. Trial ROM and an assessment of intraoperative joint stability, as well as level of patient demand, quality of bone, and the patient's general medical status can determine the final decision regarding the acceptable cup orientation in any individual case.

For example, in a recent study, the mean intraoperative abduction angle was 39 degrees (range, 23-52 degrees) with 95% of cases between 28-50 degrees. The mean postoperative abduction angle was 40 degrees (range, 25-55 degrees) with 97% (164) between 28-50 degrees. Of the 3% (6 hips) outside this range, all were within 3 degrees of their corresponding intraoperative film. 90% of postoperative films measured within 3 degrees of intraoperative films. Although 10% (17 hips) were found to be different by 4 to 14 degrees, they had an abduction angle range of 34-50 degrees (mean 43 degrees). Further analysis of the cases showing these outlier measurements identified inlet ratio deviations between intra- and postoperative radiographs of 15-22%, 23-28%, and 32% to result in cup inclination difference of 4-6 degrees, 9-11 degrees, and 14 degrees, respectively.

That is, the cup inclination measured on a radiograph showed a high ratio (closer to a circle) inlet view determined to be lower in comparison to the same cup measured on a low ratio (more oval) inlet view. Moreover, from the observations of these outliers, a correlation between the cup inclination difference (d_{ci}) and the inlet ratio deviation (dᵢᵣ) may be derived to calculate a tilt correction factor (f_{tc}). When applying the tilt correction factor, all postoperative radiographic were in a range anticipated by the intraoperative measurements.

Intraoperative digital radiography appears therefore to provide a reliable technique for assessing acetabular component abduction orientation angle during THA. That is, during surgery, the intraoperative radiograph might, for a number of reasons, not be taken from the exact same orientation as that of the preoperative X-ray. As a result, the patient's pelvis in the intraoperative radiograph may be rotated about the major axis of the pelvic inlet as compared to that of the preoperative X-ray. In such cases, the system can utilize the minor diameter or axis length or this ratio between the minor and major axes of the pelvic inlet to calculate for the surgeon an appropriate offset adjustment for the abduction angle. The surgeon then applies this angular adjustment to alter the surgeon's initial target abduction angle 753 for the patient.

The surgeon may then actuate a measurement tool 740 from the checklist 704 to display an offset line. The offset line is displayed as a line parallel to the trans-ischial line 715 and/or the teardrop line 717. The offset line can be set to start at any predetermined length, for example, the measured amount on the preoperative X-ray. This is, generally speaking, the amount of late realization of the femur in relation to the pelvis, which is as important as limb length in THA. The offset line can then be positioned at the proper location to determine the offset.

With the offset line, major and minor diameters of acetabular component can be determined. These numbers feed into a calculation of “acetabular anteversion,” another important parameter in THA.

Once all of the parameters have been adjusted accordingly, the surgeon can complete the surgical procedure. When the patient returns for evaluation, the same procedures as discussed above can be performed to measure the various parameters.

Actuation of an annotation tool permits annotation of accuracy of femoral component sizing. All annotations can be saved as part of the patient's medical record. Once the combination of these steps are complete, the surgeon is in a much better position to accurately place the acetabular component and assure that the location of the screws is acceptable. By performing this combination of steps from the checklist and getting immediate results during the actual surgical procedure, the surgeon is able to perform the surgery more accurately and quicker than without the checklist. Other parameters to be checked may be determined by the surgeon as needed.

Specific regions of interest can also be isolated and that region of interest modified in the ways described above. For example, the user can click on the image 500 and create a box 612 around the region of interest to display a blow-up of the region of interest.

In another example, the optimal acetabular cup positioning can be reliably achieved by a standard calculated by an algorithm. The surgeon can first superimpose on the radiographic image 500b what the appearance of the cup opening 792 should be. That is, in such an embodiment of the present invention, an algorithm may generate an image of the ellipse 790 that would be created on the two-dimensional radiographic image 500b of an optimally positioned acetabular cup in three-dimensional space (the standard). The surgeon may specify the desired angle of inclination I and anteversion angle A based on the patient's age, size, etc. Based on these inputs and the orientation of the radiographic images as discussed above, the algorithm generates an ellipse 790 depicting the ideal appearance of opening 792 of the acetabular cup from the perspective of the radiographic image detector when the acetabular cup has been positioned in the optimal orientation.

The ellipse 790 is then placed over the radiographic image 500 of the hip as shown in FIG. 7F to determine whether any adjustments need to be made. If so, the surgeon then adjusts the cup position to align the opening 792 of the cup so as to coincide as completely as possible with the ellipse 790. Another radiographic image may be taken to check if the proper adjustment has been made. To ensure the image is not rotated, the system allows the surgeon to determine the angle of intersection 757 between the inter-teardrop line 717 and the ilioischial line 723 on the pre-op radiographic image. The angle of intersection 757 of the two lines will then be entered on the intra-op radiographic image to rotate this image to match the pre-op image. This will ensure that the horizontal line across the screen (0 degrees) is parallel to the inter-tear drop line 717 which is critical to determine the abduction angle of the cup.

With reference to FIG. 7G, therefore, the ellipse 790 is generated using the following formulas.

Formula 1. Given the desired anteversion angle A, desired angle of inclination I, and the major diameter of the ellipse D, as input by the surgeon, the minor diameter of the ellipse d, may be calculated using the following Formula 1:

d=sin I×sin A×D

Formula 2. And, E, the angle of the ellipse's major diameter to the horizontal (as measured in the plane of the radiographic image) may be calculated using the following Formula 2:

$$E = {sin}^{- 1} ( \sqrt{\frac{( \frac{{d}^{2}}{{sin}^{2} ( A )} - {d}^{2} )}{{D}^{2} - {d}^{2}}} )$$

By way of another example, one embodiment of the system allows the surgeon to overlay an intra-op image on top of the pre-op image. The surgeon may also overlay an intra-op image on top of the intra-op image of the non-operative side. The surgeon is allowed to perform these fine adjustments (rotation, panning, etc.) to find the ideal overlay position providing the best overlap between the hips. The surgeon can also make measurements of limb length and offset discrepancy on the overlaid images. And, the foregoing image analysis algorithm may be used to overlay the images automatically.

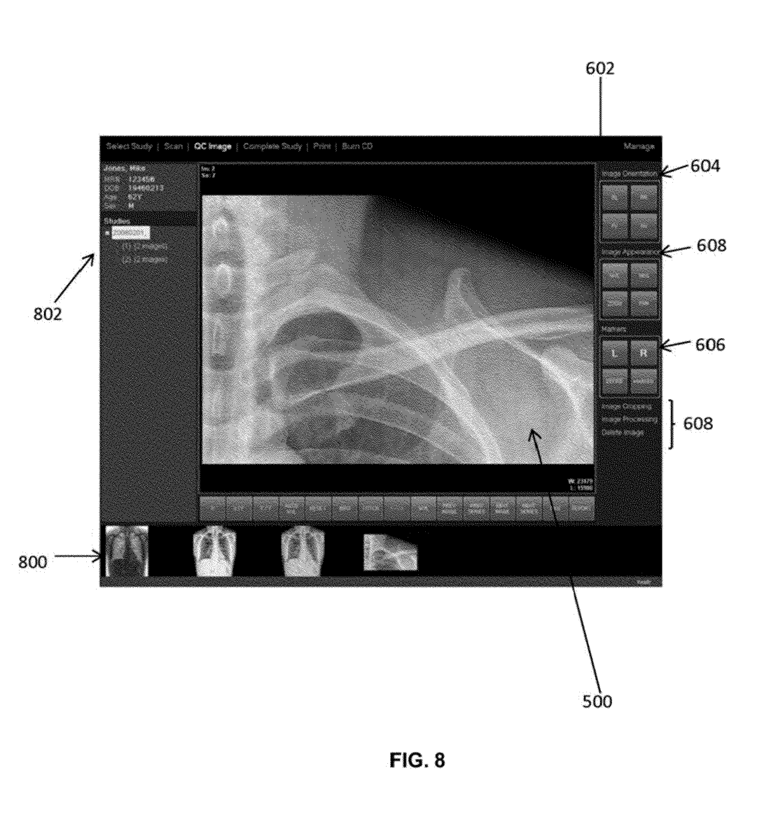

Multiple images may be acquired and saved as a series of a study. Selected images can be displayed together or individually in the image frame 204. All images in the series may be provided as thumbnail images 800 adjacent to the image frame 204 to show all images related to the image 500 displayed in the image frame 204 as shown in FIG. 8.

Once the image 500 has been acquired, before, during, or after any processing, the system can provide a checklist of parameters for the user to review and indicate whether the necessary steps have been performed. The checklist can include, but is not limited to, the following items or parameters: orientation of the radiographic image, component orientation, cup apposition (in-growth), cup anteversion angle, cup abduction angle, screw positions, femoral sizing, femoral component alignment, limb length, and offset between the first edge of a bone and a second edge of the bone.

Additional buttons may be provided to delete the image, save the image, clear the image, undo an action, redo an action, and the like. Each of these steps can be done during the operation without the surgeon having to leave his patient.

Saved files can be opened in the typical manner from a database or directory 802. The system may display a worklist window for the user to view and select study from a worklist. The worklist may be organized by a specific filter, such as name, date, medical condition, and the like. Selection of a specific filter displays all studies categorized under that filter. Each study may have additional information associated with it. The list of studies may be sortable based on any of the additional information. Selection of a study displays an image window that allows the surgeon to review acquired digital radiographic images.

Any created study can be transmitted to another computer 102a, 102b via a local area network 104 and/or the Internet 106, saved to a hard drive or saved to any other type of non-transitory computer readable medium, such as a CD, DVD, USB drive, and the like.

Additional workflow states include the state of arrival of a study, a verification state to indicate that a study is complete and accurate, a dictated state to indicate a report for a study has been dictated, a transcribed state to indicate that a report has been transcribed, and a finalized state to indicate that a report has been approved and finalized.

This system allows the user to take an X-ray before and during the middle of an operation and make the necessary adjustments immediately upon acquiring the results to greatly improve the accuracy of the surgical procedure. In addition, the accuracy resulting from each step synergistically improves the accuracy of any subsequent step and, therefore, significantly improves the outcome of the total surgical procedure in a way that cannot be achieved by improving the accuracy of any one step alone.

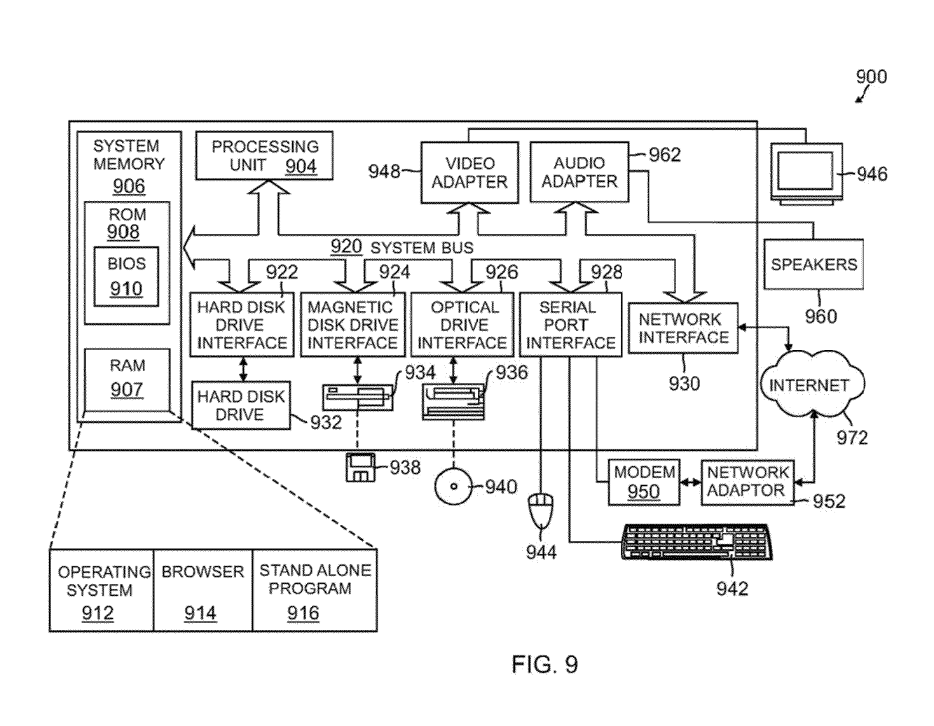

FIG. 9 depicts exemplary hardware for a surgical method and workflow system for providing efficient acquisition and processing of radiographic images for surgery. The system may be in the form of a computer 900 that includes a processing unit 904, a system memory 904, and a system bus 920 that operatively couples various system components, including the system memory 906 to the processing unit 904. There may be only one or there may be more than one processing unit 904, such that the processor of computer 900 comprises a single central processing unit (CPU), or a plurality of processing units, commonly referred to as a parallel processing environment. The computer 900 may be a conventional computer, a distributed computer, a web server, a file server, a tablet or iPad, a smart phone, or any other type of computing device.

The system bus 920 may be any of several types of bus structures including a memory bus or memory controller, a peripheral bus, a switched fabric, point-to-point connections, and a local bus using any of a variety of bus architectures. The system memory 906 may also be referred to as simply the memory, and includes read only memory (ROM) 908 and random access memory (RAM) 907. A basic input/output system (BIOS) 910, containing the basic routines that help to transfer information between elements within the computer 900, such as during start-up, is stored in ROM 908. The computer 900 may further include a hard disk drive 932 for reading from and writing to a hard disk, not shown, a magnetic disk drive 934 for reading from or writing to a removable magnetic disk 938, and/or an optical disk drive 936 for reading from or writing to a removable optical disk 940 such as a CD-ROM or other optical media.

The hard disk drive 932, magnetic disk drive 934, and optical disk drive 936 may be connected to the system bus 920 by a hard disk drive interface 922, a magnetic disk drive interface 924, and an optical disk drive interface 926, respectively. The drives and their associated computer-readable media provide nonvolatile storage of computer-readable instructions; data structures, e.g., a catalog and a context-based index; program modules, e.g., a web service and an indexing robot; and other data for the computer 900. It should be appreciated by those skilled in the art that any type of computer-readable media that can store data that is accessible by a computer, for example, magnetic cassettes, flash memory cards, USB drives, digital video disks, RAM, and ROM, may be used in the exemplary operating environment.

A number of program modules may be stored on the hard disk 932, magnetic disk 934, optical disk 936, ROM 908, or RAM 907, including an operating system 912, browser 914, stand-alone program 916, etc. A user may enter commands and information into the personal computer 900 through input devices such as a keyboard 942 and a pointing device 944, for example, a mouse. Other input devices (not shown) may include, for example, a microphone, a joystick, a game pad, a tablet, a touch screen device, a satellite dish, a scanner, a facsimile machine, and a video camera. These and other input devices are often connected to the processing unit 904 through a serial port interface 928 that is coupled to the system bus 920, but may be connected by other interfaces, such as a parallel port, game port or a universal serial bus (USB).

A monitor 946 or other type of display device is also connected to the system bus 920 via an interface, such as a video adapter 948. In addition to the monitor 946, computers typically include other peripheral output devices, such as speakers 960 connected to the system bus 920 via an audio adapter 962, and printers. These and other output devices are often connected to the processing unit 904 through the serial port interface 928 that is coupled to the system bus 920, but may be connected by other interfaces, such as a parallel port, game port, or a universal serial bus (USB).

The computer 900 may operate in a networked environment using logical connections to one or more remote computers. These logical connections may be achieved by a communication device coupled to or integral with the computer 900; the application is not limited to a particular type of communications device. The remote computer may be another computer, a server, a router, a network personal computer, a client, a peer device, or other common network node, and typically includes many or all of the elements described above relative to the computer 900, although only a memory storage device has been illustrated in FIG. 9. The computer 900 can be logically connected to the Internet 972. The logical connection can include a local area network (LAN), wide area network (WAN), personal area network (PAN), campus area network (CAN), metropolitan area network (MAN), or global area network (GAN). Such networking environments are commonplace in office networks, enterprise-wide computer networks, intranets and the Internet, which are all types of networks.

When used in a LAN environment, the computer 900 may be connected to the local network through a network interface or adapter 930, which is one type of communications device. When used in a WAN environment, the computer 900 typically includes a modem 950, a network adapter 952, or any other type of communications device for establishing communications over the wide area network. The modem 950, which may be internal or external, is connected to the system bus 920 via the serial port interface 928. In a networked environment, program modules depicted relative to the personal computer 900, or portions thereof, may be stored in a remote memory storage device. It is appreciated that the network connections shown are exemplary and other means of, and communications devices for, establishing a communications link between the computers may be used.

The system can take the form of a computer program product 916 accessible from a computer-usable or computer-readable medium providing program code for use by or in connection with a computer or any instruction execution system. For the purposes of this description, a computer-usable or computer readable medium can be any apparatus that can contain, store, communicate, propagate, or transport the program for use by or in connection with the instruction execution system, apparatus, or device.

The medium can be an apparatus or device that utilizes or implements electronic, magnetic, optical, electromagnetic, infrared signal or other propagation medium, or semiconductor system. Examples of a computer-readable medium comprise a semiconductor or solid-state memory, magnetic tape, a removable computer diskette, a random access memory, a read-only memory, a rigid magnetic disk and an optical disk. Current examples of optical disks comprise compact disk-read only memory (CD-ROM), compact disk-read/write (CD-R/W) and DVD formats.

A data processing system suitable for storing and/or executing program code comprises at least one processor coupled directly or indirectly to memory elements through a system bus. The memory elements can include local memory employed during actual execution of the program code, bulk storage, and cache memory that provide temporary storage of at least some program code in order to reduce the number of times code is retrieved from bulk storage during execution.

Input/output or I/O devices (including, but not limited to, keyboards, displays, pointing devices, etc.) can be coupled to the system either directly or through intervening I/O controllers.

Network adapters may also be coupled to the system to enable the data processing system to become coupled to other data processing systems or remote printers or storage devices through intervening private or public networks. Modems, cable modems and Ethernet cards are just a few of the currently available types of network adapters.

Furthermore, computers and other related electronic devices can be remotely connected to either the LANs or the WAN via a digital communications device, modem and temporary telephone, or a wireless link. It will be appreciated that the Internet comprises a vast number of such interconnected networks, computers, and routers.

The foregoing description of the preferred embodiment of the invention has been presented for the purposes of illustration and description. It is not intended to be exhaustive or to limit the invention to the precise form disclosed. Many modifications and variations are possible in light of the above teaching. It is intended that the scope of the invention not be limited by this detailed description, but by the claims and the equivalents to the claims appended hereto.

1. A method of intraoperative imaging, comprising:
a. capturing a radiographic image using low radiation levels;
b. adding markings to the radiographic image;
c. collecting information taken from the radiographic image and transmitting the information to a surgeon while the surgeon is conducting an operation; and
d. presenting to the surgeon, while the surgeon is conducting the operation, a checklist of a plurality of parameters associated with the measurements taken from the radiographic image on a display device.

2. The method of claim 1, further comprising taking a measurement of an anatomical structure depicted in the radiographic image.

3. The method of claim 1, further comprising making an adjustment to a surgical procedure based on the measurement.

4. A method of conducting an operation, comprising:
a. initiating the operation;
b. acquiring an intra-op radiographic image of the patient during the operation to determine an intra-op position of the patient; and
c. comparing the intra-op radiographic image with a standard to determine whether the intra-op position of the patient matches a desired position of the patient at a surgical step.

5. The method of claim 4, wherein if the intra-op position does not match the pre-op position,
a. making any adjustments to the patient;
b. acquiring a second intra-op radiographic image; and
c. comparing the second intra-op radiographic image with the standard to determine if the intra-op position matches the desired position.

6. The method of claim 4, wherein anatomical structures shown in the intra-op radiographic image are marked with first markings.

7. The method of claim 6, wherein the standard comprises second markings.

8. The method of claim 7, wherein whether the intra-op position of the patient matches the desired position of the patient at the surgical step is determined by comparing the first markings with the second markings.

9-12. (canceled)

13. A method of improving surgical workflow of an operation, comprising:
a. acquiring an intra-op radiographic image of the patient during the operation to determine an intra-op position of the patient;
b. progressing through a checklist of procedures specific to the operation;
c. comparing an anatomical structure displayed on the intra-op radiographic image with a standard to determine whether the intra-op position of the patient matches a desired position of the patient at a surgical step; and
d. if the anatomical structure does not match the standard, then adjusting the patient, acquiring a second intra-op radiographic image, and comparing the anatomical structure displayed on the second intra-op radiographic image with the standard, whereby the surgical workflow is improved.

14. The method of claim 13, wherein measurements are taken of the anatomical structure displayed on the intra-op radiographic image.

15. The method of claim 14, wherein derivatives are used to determine whether the measurements match the standard.

16. The method of claim 14, wherein the surgeon is alerted by a rating system as to the accuracy of the measurement.

17. The method of claim 13, wherein an image in a coronal plane is used to verify any positions.

18. The method of claim 13, wherein intra-op radiographic image is taken with a C-arm.

19. The method of claim 13, wherein the intra-op radiographic image undergoes a templating process to account for scaling artifacts.

20. The method of claim 19, wherein the intra-op radiographic image undergoes an edge detection process.
